(19) United States
(12) Patent Application Publication
(10) Pub. No.: US 2008/0025870 A1
(43) Pub. Date: Jan. 31, 2008

(54) Method and apparatus for treating items

(76) Inventors: Cornelis H. Groenewegen, Middletown, OH (US); Kenneth J. Roach, Hamilton, OH (US)

Correspondence Address: JohnsonDiversey, Inc., 8310 16TH STREET, M/S 509, PO BOX 902, STURTEVANT, WI, 53177-0902, US

(21) Appl. No.: 11/493,989

(22) Filed: Jul. 27, 2006

(51) Int. Cl. A61L 2/18

(52) U.S. Cl. 422/37; 23/293 R

(57) ABSTRACT

One aspect of the present invention relates to separating a gas, such as chlorine dioxide, from a solution. In another aspect of the invention, the separated gas is then used for the sterilization or sanitation of items, such as food, food storage containers, other food contact surfaces, medical devices, and the like. In yet another aspect of the invention, that gas is then decomposed or otherwise neutralized after contacting the item. In yet another aspect of the invention, the separated gas is dissolved or otherwise introduced into a pure solution, such as water, to form highly pure solution containing the gas.

### BACKGROUND OF THE INVENTION

Chlorine dioxide (ClO₂) is a strong oxidizing and antimicrobial agent. It has been reported to effectively inactivate bacteria, including pathogens, viruses, bacterial spores, and algae. In the food industry, chlorine dioxide has been used to sanitize food contact surfaces and food surfaces in the form of chlorine dioxide gas or a chlorine dioxide aqueous solution. For example, aqueous chlorine dioxide solutions have been approved for use in washing fruits and vegetables in a manner that residual chlorine dioxide does not exceed 3 ppm. Gaseous chlorine dioxide is also known as a disinfectant especially related to use in the medical sciences.

Prevention of foods from spoilage or contamination with microorganisms, including spoilage bacteria, yeast, molds, and pathogenic bacteria, has been a large challenge for the food industry. Currently, steam or aqueous sanitizers are widely used to sanitize food storage containers in the food industry. For example, some current methods of sanitizing containers on a filling line introduce peracetic acid (1000-2500 ppm), peracetic acid in steam (up to 2000 ppm stock solution), or hydrogen peroxide in steam (up to 200 ppm) in containers. After sterilization of containers, a large volume of waste solution must be stored or disposed of in appropriate manner. Further, the containers may also need to be rinsed (creating more waste solution) and subsequently dried. Also, in some applications, by-products of the sanitizing solution can be of concern.

### SUMMARY OF THE INVENTION

One embodiment of the present invention provides method and apparatus for treating, sanitizing, and/or sterilizing an item, such as a container for food, a medical device, and the like. In one embodiment of the invention, a water soluble gas, such as chlorine dioxide gas, is removed from a solution containing the water soluble gas and is circulated about the item for a time. The water soluble gas can be removed from the solution many ways. For example, it can be heated, shaken or otherwise agitated, sprayed, and the like to cause the water soluble gas to disassociate from the solution. In one particular embodiment, a vacuum is applied to the solution to remove the gas from the solution. Once the gas has contacted the item for a sufficient time, it can then be eliminated from contact with the item. In one particular embodiment, an ultraviolet light is utilized to decompose or deactivate the chlorine dioxide gas.

Another embodiment of the present invention is directed toward a process of producing a highly pure water soluble gas solution, such as chlorine dioxide gas solution. In such embodiments, the water soluble gas is removed from an impure solution and then dissolved in a pure solution. In one particular embodiment, a vacuum is used to extract chlorine dioxide gas from a solution having impurities, such as by-products from the production of the chlorine dioxide. Then, the chlorine dioxide gas is dissolved in a purified water solution.

One particular embodiment is directed toward a method for treating a food container. The method includes providing a chlorine dioxide containing solution; extracting chlorine dioxide gas from the chlorine dioxide solution with a vacuum; removing the chlorine dioxide gas from the vacuum; and injecting the chlorine dioxide gas from the vacuum into the food container. Some embodiments further provide eliminating the chlorine dioxide gas from the container, such as by exposing the chlorine dioxide gas to an ultraviolet light to deactivate the chlorine dioxide gas. Also, some embodiments indicate that the step of extracting chlorine dioxide gas from the chlorine dioxide solution with a vacuum comprises: providing a sample of the chlorine dioxide containing solution into a chamber; and applying a vacuum to the sample. The step of applying a vacuum can include sealing the chamber and actuating a piston within the chamber to generate a vacuum within the chamber.

Another particular embodiment is directed toward an apparatus for treating a food container. The apparatus comprises a reservoir containing a chlorine dioxide solution; a vacuum coupled to the reservoir to remove chlorine dioxide gas from the chlorine dioxide solution; and a conduit coupled to the vacuum and positioned to inject the chlorine dioxide gas into the container. Some embodiments further provide a device positioned adjacent the container to eliminate the chlorine dioxide gas in the container once the container has been effectively treated by the chlorine dioxide gas. That device can be an ultraviolet light adapted to be selectively illuminated to deactivate the chlorine dioxide gas in the container. The vacuum described above can include a selectively sealable chamber adapted to receive a sample of the chlorine dioxide solution and a piston selectively moveable within the chamber, wherein movement of the piston in a first direction generates a vacuum within the chamber to separate chlorine dioxide gas from the solution.

Yet another particular embodiment is directed toward a method for treating a food container. The method includes providing a food container; injecting chlorine dioxide gas into the food container to treat the food container; allowing the chlorine dioxide gas to contact the container for a sufficient time to treat the container; and deactivating the chlorine dioxide gas within the containers via an illuminated ultraviolet light positioned adjacent the container. This method can include providing a chlorine dioxide containing solution; and extracting chlorine dioxide gas from the chlorine dioxide solution with a vacuum. The step of extracting chlorine dioxide gas from the chlorine dioxide solution with a vacuum can include providing a sample of the chlorine dioxide containing solution into a chamber and applying a vacuum to the sample. The step of applying a vacuum can include sealing the chamber and actuating a piston within the chamber to generate a vacuum within the chamber.

One particular embodiment is directed toward an apparatus for treating a food container. The apparatus includes a source of chlorine dioxide gas, a conduit coupled to the source of chlorine dioxide gas and positioned to inject the chlorine dioxide gas into the container, and an ultraviolet light positioned adjacent to the container and adapted to be selectively illuminated to deactivate the chlorine dioxide gas in the container. The source of chlorine dioxide gas can include a reservoir containing a chlorine dioxide solution and a vacuum coupled to the reservoir to remove chlorine dioxide gas from the chlorine dioxide solution. The vacuum of this embodiment can include a selectively sealable chamber adapted to receive a sample of the chlorine dioxide solution and a piston selectively moveable within the chamber, wherein movement of the piston in a first direction generates a vacuum within the chamber to separate chlorine dioxide gas from the solution.

Another particular embodiment is directed toward a method of separating chlorine dioxide gas from a solution containing chlorine dioxide. The method includes providing a chlorine dioxide containing solution; providing a sample of the chlorine dioxide containing solution into a chamber; sealing the chamber; actuating a piston within the chamber to generate a vacuum within the chamber; and extracting chlorine dioxide gas from the chlorine dioxide solution with a vacuum.

Yet another particular embodiment is directed toward an apparatus for separating chlorine dioxide gas from a solution containing chlorine dioxide. The apparatus includes a reservoir containing a chlorine dioxide solution, a selectively sealable chamber adapted to receive a sample of the chlorine dioxide solution from the reservoir, and a piston selectively moveable within the chamber. Movement of the piston in a first direction generates a vacuum within the chamber to separate chlorine dioxide gas from the solution and movement of the piston in the opposite direction forces the chlorine dioxide gas from the chamber.

One other particular embodiment is directed toward a method for disinfecting an item. The method comprises providing a solution containing a water soluble gas having disinfecting properties; extracting the water soluble gas from the solution with a vacuum; removing the water soluble gas from the vacuum; injecting the water soluble gas from the vacuum toward the item; and disinfecting the item with the water soluble gas. The step of extracting the water soluble gas from the solution with a vacuum can include providing a sample of the solution into a chamber and applying a vacuum to the sample. The step of applying a vacuum can include sealing the chamber and actuating a piston within the chamber to generate a vacuum within the chamber. The step of removing the water soluble gas from the chamber can include actuating the piston within the chamber to expel the gas via an outlet in the chamber.

Another particular embodiment is directed toward an apparatus for disinfecting an item. The apparatus includes a reservoir containing a solution containing a water soluble gas having disinfecting properties, a vacuum coupled to the reservoir to remove the water soluble gas from the solution, and a conduit coupled to the vacuum and positioned to inject the water soluble gas into contact with the item to disinfect the item.

Yet another particular embodiment is directed toward a method for disinfecting an item. The method includes providing a solution containing a water soluble gas having disinfecting properties; extracting the water soluble gas from the solution with a vacuum; removing the water soluble gas from the vacuum; dissolving the extracted water soluble gas into purified water to create a highly pure solution; contacting the item with the highly pure solution; and disinfecting the item with the highly pure solution.

One particular embodiment is directed toward a method for creating a purified chlorine dioxide solution. The method comprises providing a solution containing chlorine dioxide gas and other impurities; extracting the chlorine dioxide gas from the solution with a vacuum; and dissolving the extracted chlorine dioxide gas into purified water to create a highly pure chlorine dioxide solution.

Further aspects of the present invention, together with the organization and operation thereof, will become apparent from the following detailed description of the invention when taken in conjunction with the accompanying drawings.

### BRIEF DESCRIPTION OF THE DRAWINGS

FIG. 1A is a schematic view of a vacuum device embodying aspects of the present invention, wherein the vacuum device is shown actuating a piston through an intake stroke.

FIG. 1B is a schematic view of the vacuum device shown in FIG. 1A, wherein the vacuum device is shown actuating the piston through a vacuum stroke.

FIG. 1C is a schematic view of the vacuum device shown in FIG. 1A, wherein the vacuum device is shown actuating the piston through a discharge stroke.

FIG. 1D is a schematic view of the vacuum device shown in FIG. 1A, wherein the vacuum device is shown actuating the piston through a purge stroke.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limited. The use of “including,” “comprising,” or “having” and variations thereof herein is meant to encompass the items listed thereafter and equivalents thereof as well as additional items. The terms “mounted,” “connected,” and “coupled” are used broadly and encompass both direct and indirect mounting, connecting and coupling. Further, “connected” and “coupled” are not restricted to physical or mechanical connections or couplings, and can include electrical connections or couplings, whether direct or indirect. Finally, as described in subsequent paragraphs, the specific mechanical configurations illustrated in the drawings are intended to exemplify embodiments of the invention. Accordingly, other alternative mechanical configurations are possible, and fall within the spirit and scope of the present invention.

The present invention includes multiple aspects that can be implemented or used independently or in combination. One aspect of the present invention relates to separating a water soluble gas, such as chlorine dioxide, from a solution. In another aspect of the invention, that gas is then used for the sterilization or sanitation of items, such as food, food storage containers, food processing equipment, other food contact surfaces, medical devices, and the like. In yet another aspect of the invention, that gas is then decomposed, deactivated, or otherwise neutralized after contacting the item.

The present invention has particular utility for treating, cleaning, disinfecting, sanitizing, and/or sterilizing an item, such as a container for food, a medical device, and the like. One particular embodiment of the invention provides a method and apparatus for the sterilization or sanitation of an item, such as an empty food container, other food contact surfaces, food items, medical devices, and the like. The invention can be practiced with relatively small food containers such as bottles, cans, cartons, and other food storage containers on a filling line that demand a sterilized, sanitary or aseptic condition or reduced microbial concentration. For instance, aseptic packaging may be desirable in some food applications for extended shelf life. However, this invention is not limited to aseptic requirements. Rather, containers or other surfaces can be treated for other reasons as well. Additionally, the invention can be adapted for use to sanitize or sterilize food processing equipment, such as pipes and large tanks or containers used for storing and transporting bulk quantities of food items or used for fermentation in any biotechnology industry, such as alcohol, beer, or pharmaceutical production.

In some particular embodiments, chlorine dioxide gas is the preferred gas for use in the sterilization or sanitation process. Although chlorine dioxide gas is the preferred gas for use with the methods and apparatuses described herein, other gases having known sterilization and/or sanitation capabilities can be used.

For embodiments utilizing chlorine dioxide, the chlorine dioxide gas can be initially produced as part of a chlorine dioxide solution and then removed from the solution. For example, in one particular embodiment, the chlorine dioxide solution can be produced according to the process taught in U.S. Patent Application Publication No. 2003/0064018, which is hereby incorporated by reference. In summary, this patent application teaches one particular process in which sodium chlorite is fed through an ion exchanger and a catalyst to quickly and efficiently generate a chlorine dioxide solution. However, as indicated above, other processes can be utilized to generate a chlorine dioxide solution. Such processes fall within the spirit and scope of the present invention.

Once a solution containing chlorine dioxide (or other gas) is generated, the chlorine dioxide can be removed from the solution containing chlorine dioxide. The chlorine dioxide can be removed from the solution many ways. For example, it can be heated, shaken or otherwise agitated, sprayed or atomized, and the like to cause the chlorine dioxide gas to disassociate from the solution. In one particular preferred embodiment, a vacuum is applied to the solution to remove the chlorine dioxide gas from the solution.

Putting a vacuum on a solution containing chlorine dioxide has been shown to reduce the amount of chlorine dioxide in the solution. Specifically, the vacuum causes the gas to be released from the solution into the atmosphere of the vacuum. By repeatedly exposing a chlorine dioxide solution to a vacuum, the levels of chlorine dioxide generally continue to reduce. Accordingly, in some embodiments, an intermittent or timed vacuum can be applied to a solution to maximize the extraction of chlorine dioxide gas from the solution.

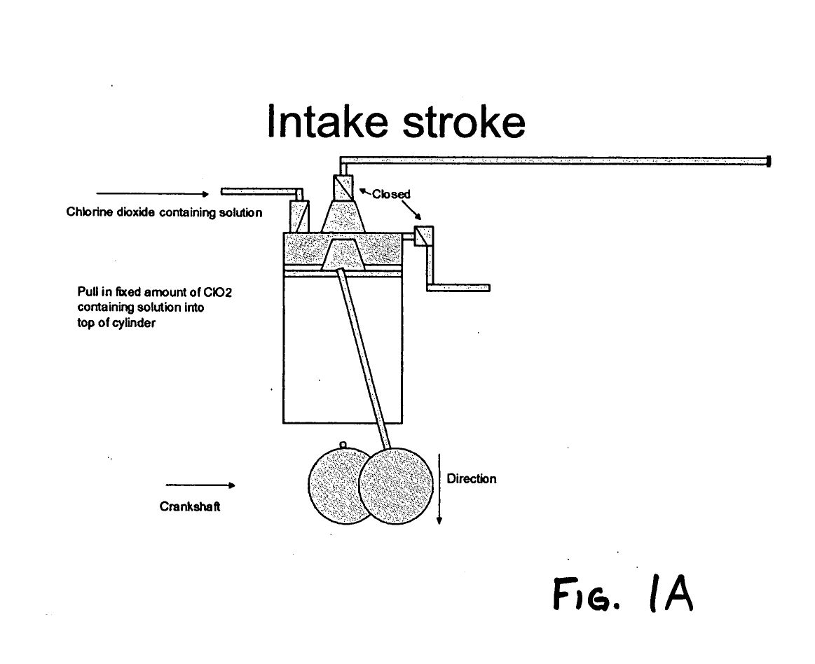

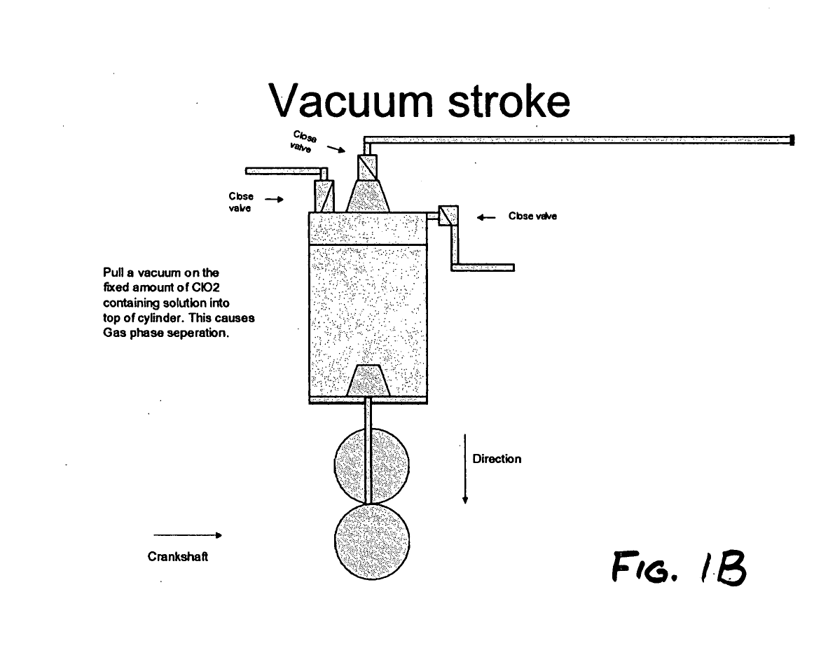

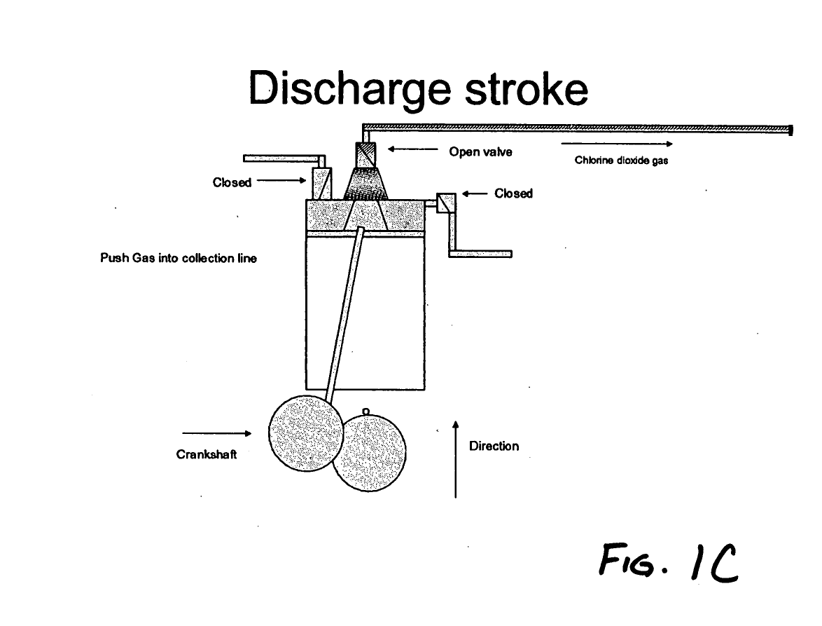

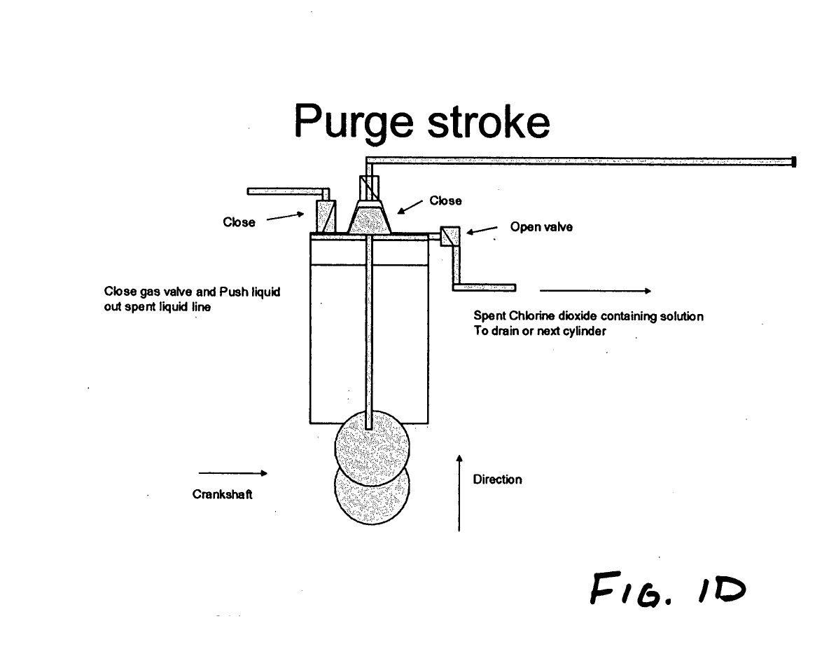

Although many different vacuum devices can be used to extract chlorine dioxide from a solution, FIG. 1 illustrates one particular vacuum device that can be utilized to generate a vacuum on a sample of chlorine dioxide solution. As shown in this figure, the vacuum device includes a chamber or cylinder having a piston that can reciprocate within the cylinder. The cylinder also includes an inlet and two outlets, with a valve located at each location to control the flow through each inlet and outlet. The inlet allows the solution containing chlorine dioxide into the cylinder. One of the outlets allows chlorine dioxide gas (that has been separated from the solution) to exit the cylinder. The other outlet allows the solution to exit the cylinder.

In operation, the vacuum has four phases of operation, as illustrated in FIGS. 1A-1D. FIG. 1A illustrates an intake stroke, wherein the piston is drawn away from the end of the cylinder adjacent the inlet and the outlets to allow the solution containing chlorine dioxide into the cylinder. As the piston is drawn back, the solution enters the cylinder via the inlet. Specifically, the valve adjacent the inlet opens to allow the solution into the cylinder. The solution can be drawn into the cylinder via the vacuum created during the back stroke or the solution can be injected with a properly timed pump. Similarly, the valve can be opened via the vacuum generated during the back stroke or the valve can be controlled via other means, such as mechanical or electrical actuators. As shown in this figure, only a small quantity (relative to the volume of the cylinder) of chlorine dioxide solution is drawn into the cylinder.

Once the chlorine dioxide solution is drawn into the cylinder via the intake stroke, the vacuum stroke illustrated in FIG. 1B begins. During the vacuum stroke, the inlet valve is closed and the piston continues to be drawn away from the end of the cylinder. Since the cylinder is sealed, this creates a vacuum within the cylinder. As this vacuum is applied to the solution, the amount of chlorine dioxide in the solution is reduced as it is released into the atmosphere within the cylinder.

Once the piston reaches the opposite end (i.e., opposite the inlet and outlets) of the cylinder, the vacuum stroke is complete and the chlorine dioxide gas discharge stroke begins. During this discharge stroke, the piston is driven toward the gas outlet and the valve located in the outlet is opened to allow the extracted chlorine dioxide gas to exit the cylinder. Once substantially all of the gas is forced out of the cylinder during this stroke, the outlet valve for the gas is closed and the outlet valve for the solution is opened (as shown in FIG. 1D). The piston continues moving toward the end of the cylinder to purge the solution from the cylinder. Once all of the solution has been purged from the cylinder, the process described in FIG. 1A can begin again.

During operation of an entire system, one or more vacuum devices can be used in series, in parallel, or in a combination of series and parallel. The number and size of vacuum devices included in an entire gas extraction system will depend upon the amount of gas needed at peak demand. Smaller operations will need fewer and/or smaller vacuum devices than larger operations, such as a larger international bottling plant.

Although the vacuum device is described specifically with reference to a chlorine dioxide solution and chlorine dioxide gas, this vacuum device can be utilized to remove other water soluble gases from a solution. For example, this may also have utility removing ozone, oxygen, ammonia, peracetic acid, and the like, from a solution.

Once the chlorine dioxide gas is extracted from the solution, the gas can be immediately used to clean, sanitize, sterilize, and/or disinfect various items or surfaces. For example, in one particular use, the chlorine dioxide gas can be injected into individual food containers, such as bottles, jars, cartons, cups, and the like. In other uses, the chlorine dioxide is injected into piping, vessels, tanks, and other food processing equipment. In yet other uses, the chlorine dioxide gas in pumped into a chamber, housing, or the like containing various items, such as food, medical equipment, and the like. In yet other embodiments, as will be described in greater detail below, the chlorine dioxide gas can be reintroduced into a pure or purified water stream to provide a highly pure chlorine dioxide solution.

Once the chlorine dioxide has contacted the item for a sufficient time to clean, disinfect, sanitize, and/or sterilize the item, the chlorine dioxide can then be eliminated from contact with the item. For example, in one particular embodiment, an ultraviolet light is utilized to decompose or deactivate the chlorine dioxide gas. In other embodiments, the gas can be evacuated by flushing the container with sterile (filtered) nitrogen, air or other suitable pressurized gas. The evacuated chlorine dioxide can then be reused, reintroduced into solution, or neutralized.

As indicated above, the chlorine dioxide gas contacts the item for a sufficient time to clean, disinfect, sanitize, and/or sterilize the item. The exact amount of contact time needed will depend upon many factors, such as the concentration of chlorine dioxide gas in the carrier gas, relative humidity adjacent the item, temperature adjacent the item or in the container, the types of target microorganisms, container surface properties (coated or uncoated), and size of the target container or item.

In one specific use, the various aspects of the present invention can be utilized in combination in a sterilization or sanitation process for relatively small food containers, such as bottles on a filling line. The vacuum device described above is used to extract chlorine dioxide gas from a reservoir or sample of chlorine dioxide solution. The chlorine dioxide gas is then injected into each individual container to disinfect the container prior to filling the container. Once the gas has contacted the surfaces for a sufficiently long time to sterilize the container, the gas can be eliminated from the container. In one specific embodiment, the gas is inactivated by an ultraviolet light. Specifically, the chlorine dioxide gas is exposed to an ultraviolet light of sufficient frequency and wave length for a sufficient period of time to deactivate or decompose the gas. In some embodiments, the gas is exposed to the ultraviolet light long enough such that the residual chlorine dioxide levels fall below the levels allowed in potable water. Some embodiments can utilize a rinse with sterile water to further remove the chlorine dioxide gas from the containers if desired or needed.

In another specific use, the various aspects of the present invention can be utilized while processing food, such as fruit, vegetables, poultry, etc. The vacuum device described above is used to extract chlorine dioxide gas from a chlorine dioxide solution. The chlorine dioxide gas is then injected into a chamber containing the food. The gas can be injected directly onto the food or indirectly onto the food via the surrounding environment. Once the gas has contacted the surfaces for a sufficiently long time, the gas can be eliminated from the chamber or the food can be removed from the chamber. In one specific embodiment, the gas is eliminated or inactivated by an ultraviolet light as described above. Subsequently, the food is removed from the chamber. Alternatively, the food can be removed from the chamber via a conveyor system and any escaping gas can be eliminated via an ultraviolet light located adjacent the exit of the chamber.

In yet other uses, the extracted water soluble gas, such as chlorine dioxide, can be injected in a room (or chamber, container, etc.) to disinfect the room or objects within the room. For example, the chlorine dioxide gas can be used to treat library books or rare art. Additionally, the gas can be used to decontaminate an entire room, such as a room with suspected anthrax and/or mold contamination.

As indicated above, the present invention can be used to create a highly pure chlorine dioxide (or other soluble gas) solution. Particularly, once the gas is extracted from the original solution, the gas can be dissolved into a pure or purified solution, such as purified water. The gas can be dissolved into the pure solution many ways known in the art. For example, in one embodiment, the gas can be dissolved in the solution by bubbling the gas into the solution.

This process of extracting a water soluble gas from one solution and dissolving it in another solution can enable one to make a highly purified solution containing the gas. For example, many chlorine dioxide solutions can contain some impurities that may be undesirable in some applications. For example, some by-products from reactions that produce chlorine dioxide solutions can include chlorate, chloride, chlorite, hypochlorite, and the like. These by-products may be a concern in some applications. Accordingly, the gas can be extracted from the solution and dissolved into purified water to create a highly pure chlorine dioxide solution.

The embodiments described above and illustrated in the figures are presented by way of example only and are not intended as a limitation upon the concepts and principles of the present invention. As such, it will be appreciated by one having ordinary skill in the art that various changes in the elements and their configuration and arrangement are possible without departing from the spirit and scope of the present invention. For example, the examples described above can be modified to apply to food processing equipment, medical devices, etc. Further, various alternatives to the certain features and elements of the present invention may be described with reference to specific embodiments of the present invention. With the exception of features, elements, and manners of operation that are mutually exclusive of or are inconsistent with each embodiment described above, it should be noted that the alternative features, elements, and manners of operation described with reference to one particular embodiment may be applicable to the other embodiments.

Various features of the invention are set forth in the following claims.

What is claimed is:

1. A method for treating a food container, the method comprising:
providing a chlorine dioxide containing solution;
extracting chlorine dioxide gas from the chlorine dioxide solution with a vacuum;
removing the chlorine dioxide gas from the vacuum; and
injecting the chlorine dioxide gas from the vacuum into the food container.

2. The method of claim 1 further comprising eliminating the chlorine dioxide gas from the container.

3. The method of claim 2 wherein the step of eliminating the chlorine dioxide gas from the container comprises exposing the chlorine dioxide gas to an ultraviolet light to deactivate the chlorine dioxide gas.

4. The method of claim 1 wherein the step of extracting chlorine dioxide gas from the chlorine dioxide solution with a vacuum comprises:
providing a sample of the chlorine dioxide containing solution into a chamber; and
applying a vacuum to the sample.

5. The method of claim 4 wherein the step of applying a vacuum comprises:
sealing the chamber; and
actuating a piston within the chamber to generate a vacuum within the chamber.

6. The method of claim 5 wherein the step of removing the chlorine dioxide gas from the chamber comprises actuating the piston within the chamber to expel the gas via an outlet in the chamber.

7. An apparatus for treating a food container, the apparatus comprising:
a reservoir containing a chlorine dioxide solution;
a vacuum coupled to the reservoir to remove chlorine dioxide gas from the chlorine dioxide solution; and
a conduit coupled to the vacuum and positioned to inject the chlorine dioxide gas into the container.

8. The apparatus of claim 7, further comprising a device positioned adjacent the container to eliminate the chlorine dioxide gas in the container once the container has been effectively treated by the chlorine dioxide gas.

9. The apparatus of claim 8, wherein the device that eliminates the chlorine dioxide gas comprises an ultraviolet light adapted to be selectively illuminated to deactivate the chlorine dioxide gas in the container.

10. The apparatus of claim 7, wherein the vacuum comprises:
a selectively sealable chamber adapted to receive a sample of the chlorine dioxide solution; and
a piston selectively moveable within the chamber, wherein movement of the piston in a first direction generates a vacuum within the chamber to separate chlorine dioxide gas from the solution and movement of the piston in the opposite direction forces the chlorine dioxide gas from the chamber.

11. A method for treating a food container, the method comprising:
providing a food container;
injecting chlorine dioxide gas into the food container to treat the food container;
allowing the chlorine dioxide gas to contact the container for a sufficient time to treat the container; and
deactivating the chlorine dioxide gas within the containers via an illuminated ultraviolet light positioned adjacent the container.

12. The method of claim 11, further comprising:
providing a chlorine dioxide containing solution; and
extracting chlorine dioxide gas from the chlorine dioxide solution with a vacuum.

13. The method of claim 11, wherein the step of extracting chlorine dioxide gas from the chlorine dioxide solution with a vacuum comprises:
providing a sample of the chlorine dioxide containing solution into a chamber; and
applying a vacuum to the sample.

14. The method of claim 13, wherein the step of applying a vacuum comprises:
sealing the chamber; and
actuating a piston within the chamber to generate a vacuum within the chamber.

15. An apparatus for treating a food container, the apparatus comprising:
a source of chlorine dioxide gas;
a conduit coupled to the source of chlorine dioxide gas and positioned to inject the chlorine dioxide gas into the container; and
an ultraviolet light positioned adjacent to the container and adapted to be selectively illuminated to deactivate the chlorine dioxide gas in the container.

16. The apparatus of claim 15, wherein the source of chlorine dioxide gas comprises:
a reservoir containing a chlorine dioxide solution; and
a vacuum coupled to the reservoir to remove chlorine dioxide gas from the chlorine dioxide solution.

17. The apparatus of claim 16, wherein the vacuum comprises:
a selectively sealable chamber adapted to receive a sample of the chlorine dioxide solution; and
a piston selectively moveable within the chamber, wherein movement of the piston in a first direction generates a vacuum within the chamber to separate chlorine dioxide gas from the solution and movement of the piston in the opposite direction forces the chlorine dioxide gas from the chamber.

18. A method of separating chlorine dioxide gas from a solution containing chlorine dioxide, the method comprising:
providing a chlorine dioxide containing solution;
providing a sample of the chlorine dioxide containing solution into a chamber;
sealing the chamber;
actuating a piston within the chamber to generate a vacuum within the chamber; and
extracting chlorine dioxide gas from the chlorine dioxide solution with a vacuum.

19. An apparatus for separating chlorine dioxide gas from a solution containing chlorine dioxide, the apparatus comprising:
a reservoir containing a chlorine dioxide solution;
a selectively sealable chamber adapted to receive a sample of the chlorine dioxide solution from the reservoir; and
a piston selectively moveable within the chamber, wherein movement of the piston in a first direction generates a vacuum within the chamber to separate chlorine dioxide gas from the solution and movement of the piston in the opposite direction forces the chlorine dioxide gas from the chamber.

20. A method for disinfecting an item, the method comprising:
providing a solution containing a water soluble gas having disinfecting properties;
extracting the water soluble gas from the solution with a vacuum;
removing the water soluble gas from the vacuum;
injecting the water soluble gas from the vacuum toward the item; and
disinfecting the item with the water soluble gas.

21. The method of claim 20 wherein the step of extracting the water soluble gas from the solution with a vacuum comprises:
providing a sample of the solution into a chamber; and
applying a vacuum to the sample.

22. The method of claim 21 wherein the step of applying a vacuum comprises:
sealing the chamber; and
actuating a piston within the chamber to generate a vacuum within the chamber.

23. The method of claim 22 wherein the step of removing the water soluble gas from the chamber comprises actuating the piston within the chamber to expel the gas via an outlet in the chamber.

24. An apparatus for disinfecting an item, the apparatus comprising:
a reservoir containing a solution containing a water soluble gas having disinfecting properties;
a vacuum coupled to the reservoir to remove the water soluble gas from the solution; and
a conduit coupled to the vacuum and positioned to inject the water soluble gas into contact with the item to disinfect the item.

25. The apparatus of claim 24, further comprising a device positioned adjacent the item to eliminate the water soluble gas once the item has been effectively treated by the water soluble gas.

26. The apparatus of claim 25, wherein the water soluble gas is chlorine dioxide and the device that eliminates the chlorine dioxide gas comprises an ultraviolet light adapted to be selectively illuminated to deactivate the chlorine dioxide gas in the container.

27. The apparatus of claim 24, wherein the vacuum comprises:
a selectively sealable chamber adapted to receive a sample of the solution; and
a piston selectively moveable within the chamber, wherein movement of the piston in a first direction generates a vacuum within the chamber to separate water soluble gas from the solution and movement of the piston in the opposite direction forces the water soluble gas from the chamber.

28. A method for disinfecting an item, the method comprising:
providing a solution containing a water soluble gas having disinfecting properties;
extracting the water soluble gas from the solution with a vacuum;
removing the water soluble gas from the vacuum;
dissolving the extracted water soluble gas into purified water to create a highly pure solution;
contacting the item with the highly pure solution; and
disinfecting the item with the highly pure solution.

29. The method of claim 28 wherein the step of extracting the water soluble gas from the solution with a vacuum comprises:
providing a sample of the solution into a chamber; and
applying a vacuum to the sample.

30. The method of claim 29 wherein the step of applying a vacuum comprises:
sealing the chamber; and
actuating a piston within the chamber to generate a vacuum within the chamber.

31. The method of claim 30 wherein the step of removing the water soluble gas from the chamber comprises actuating the piston within the chamber to expel the gas via an outlet in the chamber.

32. A method for creating a purified chlorine dioxide solution, the method comprising:
providing a solution containing chlorine dioxide gas and other impurities;
extracting the chlorine dioxide gas from the solution with a vacuum; and
dissolving the extracted chlorine dioxide gas into purified water to create a highly pure chlorine dioxide solution.
